(19) United States
(12) Patent Application Publication
(10) Pub. No.: US 2010/0049039 A1
(43) Pub. Date: Feb. 25, 2010

(54) System and Method of Treating Skin Imperfections Including a Diagnostic Tool

(76) Inventor: Charles R. Heehler, Port Orange, FL (US)

Correspondence Address: McKinney Law, LLC, 121 S. Orange Ave, Suite 1500, Orlando, FL, 32801, US

(21) Appl. No.: 12/615,797

(22) Filed: Nov. 10, 2009

Related U.S. Application Data
(63) Continuation-in-part of application No. 11/007,904, filed on Dec. 8, 2004.

(51) Int. Cl. A61B 6/00; A61B 5/103

(52) U.S. Cl. 600/425; 600/587

(57) ABSTRACT

A system and method of treating skin imperfections including a diagnostic tool is disclosed. In a particular embodiment the system includes a diagnostic tool to scan a preselected area of skin of a client to detect at least one skin imperfection and an associative module to associate a skin value to the client. Further, the system includes that a first unadulterated ingredient of alpha lipoic acid, dimethylaminoethenol, vitamin A, vitamin C, and vitamin E, or any combination thereof, is selected based on the skin value, and a second unadulterated ingredient of retinol, alpha and beta hydroxyl acids, or any combination thereof, is selected based on the skin value. In addition, a customized product is compounded using selected first and second unadulterated ingredients and the product is compounded manually by a technician at a point of sale.

### I. CROSS-REFERENCE TO RELATED APPLICATIONS

This application is a continuation in part of U.S. patent application Ser. No. 11/007,904 filed Dec. 8, 2004.

### II. FIELD

The present disclosure is generally related to a system and method of treating skin imperfections.

### III. DESCRIPTION OF RELATED ART

Skin care products have been known for hundreds of years. Until recently, such products were mainly moisturizers and emollients. Since the 1970's, research and development in the skin care industry has progressed with the discovery of nutrients which can treat the cause of unhealthy skin: anti-oxidants such as alpha lipoic acid and dimethylaminoethanol, vitamins A, C ester and E, defoliants such as retinol and alpha and beta hydroxyl acids, bio-engineered substances which stimulate collagen and elastin syntheses, protein enzyme suppressants, and other anti-aging substances, along with newly discovered vehicles which can penetrate the skin. However, when products incorporating these ingredients are prepared for mass-marketing, they usually incorporate preservatives and stabilizers so that they have a long shelf life. The preservatives and stabilizers tend to degrade the therapeutic efficacy of many of the active ingredients, and to interfere with their absorption. Also, mass production techniques may expose the active ingredients to temperature extremes which can further reduce their effectiveness.

Mass-marketing of skin care products on the internet and in discount stores such as Wal-Mart®, Target® and K-Mart® has drawn business away from the traditional cosmetic departments of upscale department stores. The cosmetic and skin care industry suffers from lack of differentiation from the mass-marketed products and needs to pursue an innovative marketing method to appeal to an upscale clientele that is concerned about pollutants and toxins in their food and consumable items, and that wants the very best that science has to offer in skin care. Importantly, the majority of skin care products that are available from cosmetic manufacturers seek to remedy skin deficiencies or covering up defects, but are not aimed at treating the cause of such deficiencies.

Accordingly, a particular advantage provided by embodiments of the system and method of treating skin imperfections is the ability to implement the system and method in upscale boutiques, specialty stores, fine department stores' cosmetic department, or a similar environment, and to compound customized skin care products from fresh, unadulterated super nutrients at the point of purchase in response to determining a value of skin texture.

Other aspects, advantages, and features of the present disclosure will become apparent after review of the entire application, including the following sections: Brief Description of the Drawings, Detailed Description, and the Claims.

### IV. SUMMARY

In a particular embodiment, a method of treating skin imperfections is disclosed.

The method includes scanning a preselected area of skin of a client using a diagnostic tool, detecting a first edge and a second edge of at least one skin imperfection within the preselected area using the diagnostic tool, detecting a surficial depth of the at least one skin imperfection using the diagnostic tool, determining a width of the at least one skin imperfection using a spatial difference between the first edge relative to the second edge, and calculating a volume of the at least one skin imperfection using the depth and width of the at least one skin imperfection. In addition, the method includes associating a skin value to the client based on a total volume of the at least one skin imperfection and a size of the preselected area of the skin of the client, manually selecting a first unadulterated ingredient from a plurality of unadulterated ingredients based on the skin value, manually selecting a second unadulterated ingredient from the plurality of ingredients based on the skin value, and compounding the first unadulterated ingredient and the second unadulterated ingredient to form a customized product at the point of sale, where the compounding is manually completed by a technician. By definition, “compounding” means to form a new whole; to make more intense; to change integrity; to be synergistic, as elements that cooperate for an enhanced effect. A portion of the customized product may be applied to an affected area of the at least one skin imperfection at the point of sale.

In a particular embodiment the diagnostic tool may include a photodetector and a laser that emits coherent light where the photodetector is responsive to the electromagnetic reflections from the skin. The diagnostic tool may be aimed at the preselected area of skin then activated by a trigger. The diagnostic tool may further include a memory for storing the skin value of the client. The skin value may be displayed to the client. The diagnostic tool may be calibrated and referenced at a start of each scanning session. The first unadulterated ingredient may be one of alpha lipoic acid, dimethylaminoethenol, vitamin A, vitamin C, and vitamin E, or any combination thereof. The second unadulterated ingredient may be one of retinol, alpha and beta hydroxyl acids, or any combination thereof.

In another particular embodiment, a system of treating skin imperfections is disclosed. The system includes a diagnostic tool to scan a preselected area of skin of a client to detect at least one skin imperfection and an associative module to associate a skin value to the client based on the at least one skin imperfection. A first unadulterated ingredient of alpha lipoic acid, dimethylaminoethenol, vitamin A, vitamin C, and vitamin E, or any combination thereof, may be selected based on the skin value. Further, a second unadulterated ingredient of retinol, alpha and beta hydroxyl acids, or any combination thereof, may be selected based on the skin value. In addition, a customized product at the point of sale is compounded using a selected first unadulterated ingredient and a selected second unadulterated ingredient, where the product is compounded manually by a technician.

In addition, the diagnostic tool may include a laser that emits coherent light to scan a preselected area of skin and a photodetector that is responsive to electromagnetic reflections by the laser that are reflected from a surface of the skin, where the electromagnetic reflections detect at least one skin imperfection. The associative module associates the skin value to the client based on the electromagnetic reflections from the at least one skin imperfection. The customized product is applied to an affected area of the at least one skin imperfection. The diagnostic tool may further include a memory for storing the skin value of the client and a display for displaying a representation of the electromagnetic reflections to the client.

In addition, the system may include a sponsor entity associated with a skin care product provider entity, where the sponsor entity may provide business-conducting space to the technician. The customized product may include at least one pure, fresh, preservative-free, anti-aging super nutrient of proven therapeutic effectiveness in topical application, and appropriate vehicles for topical delivery of the super nutrients.

The system may also include means to compare the electromagnetic reflections to previously stored data for the client to determine results of treatment using the customized product. The diagnostic tool may be configured to detect a surficial depth of the at least one skin imperfection based on a known speed of the coherent light and measuring a time interval between an initial signal received representing a normal surface of the skin, and a subsequent signal received representing a depth of the skin imperfection.

Other aspects, advantages, and features of the present disclosure will become apparent after review of the entire application, including the following sections: Brief Description of the Drawings, Detailed Description, and the Claims.

### V. BRIEF DESCRIPTION OF THE DRAWINGS

FIG. 1 is a flow diagram of a particular embodiment of a method of treatment of skin imperfections;

FIG. 2 is a diagram of a particular embodiment of a system of treatment of skin imperfections;

FIG. 3 is a diagram of a portion of a client's skin showing a representation of skin imperfections to be treated;

FIG. 4 is a diagram showing an exaggerated scale of a skin imperfection of the client and a schematic of the laser and photodetector of the present method and system;

FIG. 5 is a block diagram of an illustrative embodiment of the diagnostic tool that may be used in the system to treat skin imperfections; and

FIG. 6 is a block diagram of an illustrative embodiment of a general computer system.

### VI. DETAILED DESCRIPTION

Uncovering the latest trends in consumer behavior reveals that many customers now have a strong desire for cosmetic products that have natural ingredients and formulations that are pure, risk free, noninvasive and do not jeopardize the recipient's sense of identity. The term “natural” is interpreted by the consumer as a preparation that is free of harmful chemicals and that contains no impurities or adulterations. Increased life spans resulting in the development of an older population, the pursuit of agelessness, technological advances, and a growing awareness of health and wellness are trends that are driving the skin care market. Moreover, research indicates that women change their makeup buying habits between the ages of 35 and 44 and turn their attention towards anti-aging, especially in the eye category where concern about wrinkles and fine lines around the eyes leads women away from eye shadow and eye liner. Research indicates that women not only change their preferences with age but they also become bigger consumers.

The skin care category is becoming increasingly more high tech with a trend toward cosmeceuticals, whose sales are expected to reach $8.2 billion by 2012. Skin care products will account for more than 63% of the total cosmeceuticals product demand in 2012, with the demand for anti-aging products growing at twice the rate of other cosmeceuticals products. This trend is expected to remain on track even as the economy struggles.

Since innovation in product efficacy is more and more the price of entry, the lines between prestige and mass brands are more and more blurred. Drug store brands are likely to attract department store shoppers looking to “trade down” from prestige products during an economic slide. The increasing availability of medical practitioners to dispense proprietary medications, not long ago considered unethical, will also have a negative influence on skin treatment sales of the prestigious department store retailer. Thus, for the prestige and ultra-prestige retailer to advance, marketing must now place greater emphasis on ways to differentiate. Differentiation that is uniquely distinctive is absolutely essential. Moreover, while positioning products as glamorous is indeed important in a fashionable environment, the unique efficacies of innovative science as the deciding factor in anti-aging treatment choices will triumph. Sensory and emotional benefits still must complete the package because ultimately, it is the benefit of the product rather than the formula that is important to the customer.

Despite the 2008 economic slump, the luxury segment of cosmetic treatment is growing. Price is not the most important purchasing criteria: price is not an issue when the product is desired. The image of the retailer, the quality and the conceptual and distinctive design of the product are central issues that influence the sale. The ultra-premium skincare product category, are those priced at $150.00 and above. These account for 3% of the $2.2 billion prestige skin care market, but generate the strongest growth in the overall category. Significant gains are expected through 2010.

Accordingly, there is a niche market for skin rejuvenation actives and anti-aging skincare that may be addressed by providing preservative-free molecules, compounded and packaged at the point-of-purchase in the cosmetic bays of the prestigious department stores, to form a clinically proven, highly efficacious and safe skin preparation for both women and men. To complete treatment for a cumulative effect and persistent result, a timely, free refill procedure may be established. The showmanship capabilities provided by the act of a technician, at the point-of-purchase fulfills the key issue to building a sustainable brand and to building a viable women's and men's cosmetic and fragrance department: education by means of an entertaining “story-tell” show, thus establishing an emotional connection to the department store patron. Moreover, “show time” whose flexible duration can be scheduled for one minute or for the duration of up to one hour or more, will not be dependent on the infrequent visit of a company demonstration representative, but rather, an everyday event, tailor-made by store management to address local customer demographics. Compounding preservative-free anti-aging skincare at the point-of-purchase is a unique method of skincare.

In one particular embodiment, the method includes selecting a first unadulterated ingredient, manually selecting a second unadulterated ingredient, and compounding the first unadulterated ingredient and the second unadulterated ingredient to form a customized product at the point of sale, where the compounding is manually completed by a technician. By definition, “compounding” means to form a new whole; to make more intense; to change integrity; to be synergistic, as elements that cooperate for an enhanced effect.

The distinctive differentiation provided by the presentation of preservative-free skin rejuvenation actives and anti-aging skin treatments and tonics, exclusively, will initiate an excellent strategy for taking advantage of a recession and for getting ahead of peers. Providing the most loyal patrons of various department stores with the distinctive qualities of skincare will test and take advantage of the traffic building potential of a new and more personalized presentation in anti-aging skincare. Offering both the female and male aspirational buyer unique motives for visiting the women's cosmetic department and men's grooming department, respectively, will provide a great variety of sales opportunities. Price is not an issue when the product is desired.

The women's cosmetic and fragrance department, and men's grooming department, have always been the cornerstone of particular upscale department stores, and are used to set the tone of the prestige retailer. Designed to address both current and future needs of loyal patrons, the cosmetic department generates enthusiasm and excitement and serves as a major traffic driver. But most importantly, the cosmetic department must convey to the customer a unique store image. This image is critical. The number one value driver for retail customers is brand and atmosphere. The core emotion that drives brand and atmosphere is creativity, which is why offering something unique and exclusive is essential.

Preservative-free, anti-aging skincare preparations, compounded at the point-of-purchase as described herein will expose the store patron to an exclusive experience defined by extraordinary efficacy through advanced scientific technology, and will serve to enhance the shopping experience. A unique, exclusive “clinical experience,” enveloped in glamour and fashion, will inoculate the customer with a feeling of euphoria and encourage the customer to “plan hopefully ahead” while making additional department color/makeup/fragrance cosmetic purchases, in addition to other related, new life-style purchases located in the convenience of adjacent designer departments and store salons.

The procedure for compounding and packaging preservative-free skincare at the point-of-purchase has never been attempted. Line identification and presentation will be designed to be inviting and irresistible, featuring state-of-the-art, communicative display work devised to educate and entertain in an upscale, fashionable manner. By definition, “compounding” means to form a new whole; to make more intense; to change integrity; to be synergistic, as elements that cooperate for an enhanced effect.

Moreover, the method and system may also entail detailing the medical profession on the new availability of preservative-free, state-of-the-art actives found exclusively at particular department stores. Importantly, plastic surgeons and dermatologists who presently have no way of obtaining preservative-free, state-of-the-art actives and only a very limited accessibility to preservative-free anti-aging skincare, will now have Neiman Marcus as a professional destination for their patients who have skin sensitivities due to the impurities of preservatives and other adulterations. Because of the very limited distribution of unique, state-of-the-art actives and because of the relatively short shelf life of preservative-free actives, it would indeed be too problematic for national drugstore chains and for the relatively few independent drugstores that offer compounding services, to stock and to provide the medical profession with unique, state-of-the-art, preservative-free actives. As it is currently, the established pharmaceutical industry does not provide dermatologists and plastic surgeons an economical and convenient professional source for accessing state-of-the-art, preservative-free actives and only a very limited source for providing preservative-free anti-aging skincare. In contrast to the professional pharmacist of a drugstore or apothecary, my esthetician /consultant will be able to address this sophisticated need successfully because the procedures for handling a relatively unstable inventory control is what the consultant is specializing in at the point-of-purchase. Both the pharmaceutical industry and the upscale cosmetic industry have a need for this new platform of distribution.

Accordingly, a variety of preservative-free, anti-oxidant/super-nutrient molecules (e.g., first and second unadulterated ingredients) are shipped to the point-of-purchase on a timely basis as inventory rotation requires. The anti-aging ingredients will then be protected at the point-of-purchase until needed. At which time, in response to a customer skin tissue evaluation, the esthetician/consultant/technician will compound a variety of actives/anti-oxidant/super-nutrient molecules with a third unadulterated ingredient as a delivery system that is deep penetrating to the deepest layer of the dermis. A sophisticated countertop-bay dispensing apparatus may be used to maintain a sterile compounding environment. It is a matter of scientific record that the only long lasting solution to the problems of unhealthy skin and the unwanted visible characteristics of aging, are those preparations that have been scientifically tested and proven to penetrate down to the deepest layers of the dermis where the unhealthy processes are actually taking place. The anti-aging skin treatments pursuant to the disclosed method and system are formulated to exact specifications so that they can penetrate deeply into the skin in a manner that, traditional skincare formulas cannot.

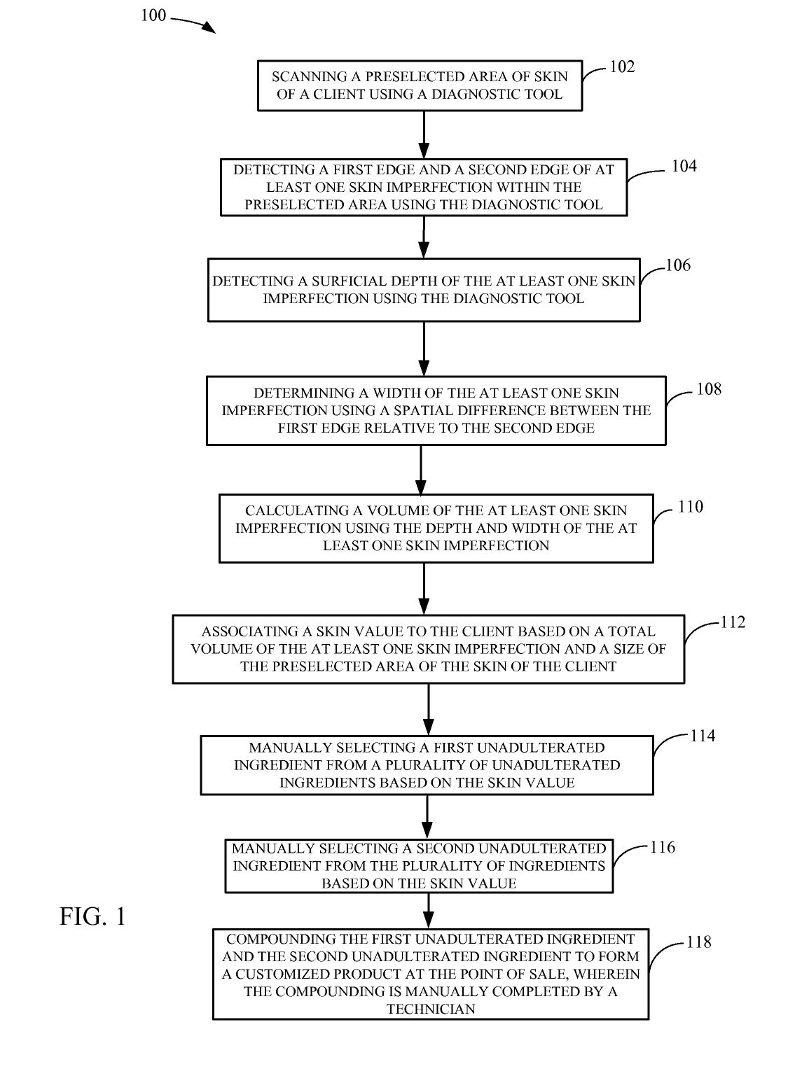

Referring to FIG. 1, a particular illustrative embodiment of a method of treating skin imperfections is disclosed and generally designated 100. A preselected area of skin of a client may be scanned using a diagnostic tool, at 102. The diagnostic tool may be one of a variety of different instruments (e.g., magnifying glass) that can detect skin imperfections. A first edge and a second edge of at least one skin imperfection is detected, at 104, within the preselected area using the diagnostic tool. Moving to 106, a surficial depth of the at least one skin imperfection is detected using the diagnostic tool. At 108, a width of the at least one skin imperfection is determined by using a spatial difference between the first edge relative to the second edge. A volume of the at least one skin imperfection is calculated using the depth and width of the at least one skin imperfection, at 110. A skin value is associated to the client, at 112, based on a total volume of the at least one skin imperfection and a size of the preselected area of skin of the client. Continuing to 114, a first unadulterated ingredient is manually selected from a plurality of unadulterated ingredients based on the skin value. A second unadulterated ingredient is manually selected, at 116, from the plurality of ingredients based on the skin value. The first unadulterated ingredient and the second unadulterated ingredient are compounded to form a customized product at the point of sale, where the compounding is manually completed by a technician, at 118. The first and second unadulterated ingredients may include alpha lipoic acid, dimethylaminoethenol, vitamin A, vitamin C, vitamin E, retinol, alpha and beta hydroxyl acids. However, this is not an exhaustive list of the possible unadulterated ingredients for use with the method, but rather is provided as exemplary ingredients. In addition, a third unadulterated ingredient may be provided, which is adapted to serve as a delivery vehicle for the first and second unadulterated ingredients to penetrate down to a deep layer of a dermis of the client.

The diagnostic tool is aimed at the client and activated by a trigger. In addition, the diagnostic tool may include a photodetector and a laser that emits coherent light. The photodetector is responsive to the electromagnetic reflections (i.e., signal) from the skin. The surficial depth of the at least one skin imperfection may be detected by using the known speed of the coherent light and measuring a time interval between an initial signal received by the photodetector representing a normal surface of the skin, and a subsequent signal received representing a depth of the skin imperfection. The cause of the at least one skin imperfection may be treated by applying a portion of the customized product to an affected area at the point of sale. In addition, the customized product may be applied at home. The diagnostic tool may be referenced and calibrated at the beginning of each scanning session. A memory of the diagnostic tool may be used for storing the current skin value and/or historical skin values for the client. Accordingly, the skin value may be displayed to the client and an improvement in the skin value may be determined based on comparing the current skin value to a past skin value.

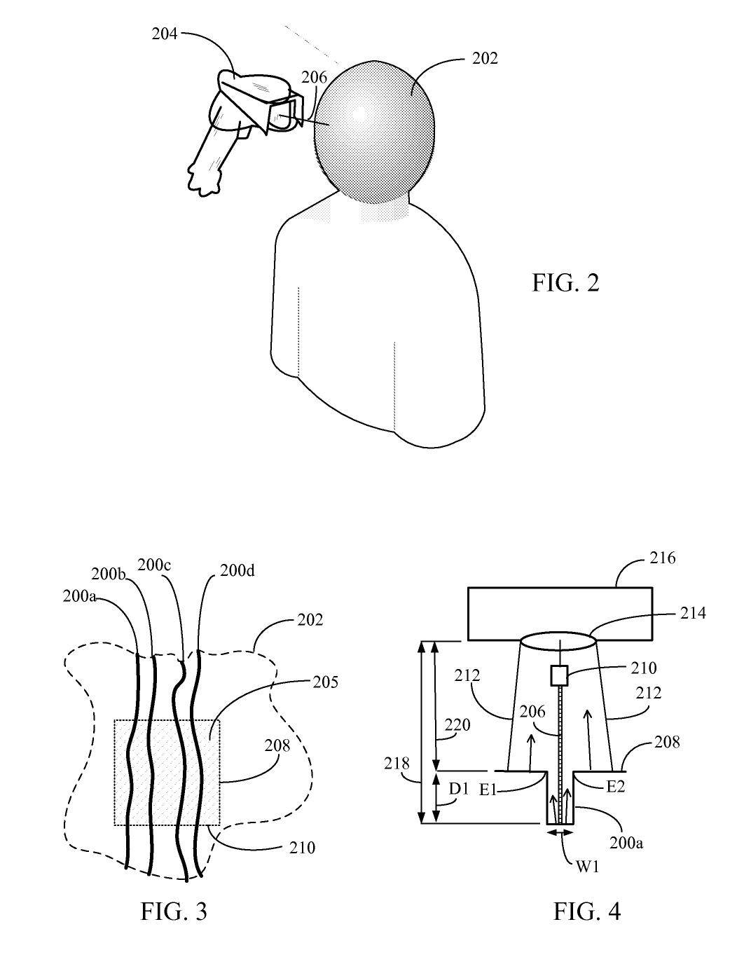

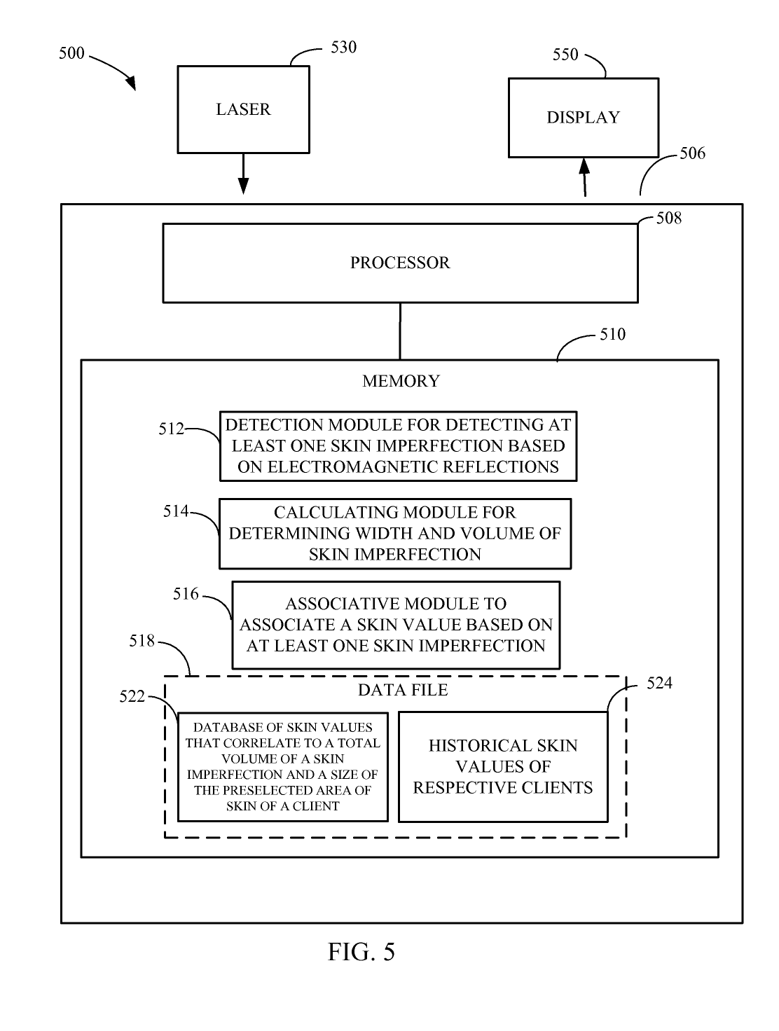

A diagram that illustrates an example of treating of skin imperfections is disclosed in FIG. 2. A diagnostic tool 204 is aimed at the skin 202 of a client. A laser beam 206 of coherent light is focused on the skin. A first skin imperfection 200a is detected and the location may be stored using an (x, y) Cartesian coordinate system or any other similar type of system that may be used to identify a spatial location of the first skin imperfection. The skin 202 may have several skin imperfections (200a, 200b, 200c, 200d) about a preselected area 205. For example, the skin imperfections may be a series of wrinkles. The preselected area 205 has a first planar dimension 208 and a second planar dimension 210. A value in square units (e.g., square inches) is calculated by multiplying the first dimension 208 by the second dimension 210.

Referring now to FIG. 3, the first skin imperfection 200a is shown exaggerated for illustrative purposes. A first edge E1 of the imperfection 200a is detected based on comparing a time interval between transmitting the coherent light 206 and the signals received by optics 214 of the photodetector 216. For example, the time interval between signals is constant until a depression causes the time interval between transmitting and receiving the signal to increase. Thus, an edge of the skin imperfection has been detected and the spatial location is then stored. The second edge E2 of the imperfection is detected and stored using the location where the time interval between transmitting and receiving the signal decreases. The width W1 of the imperfection (e.g. wrinkle) is then calculated using the spatial difference between the first edge E1 and the second edge E2. The depth D1 of the imperfection 200a is calculated by the difference of the distance 220 between the photodetector 216 and normal surface of the skin 208 and the distance 218 between the photodetector 216 and the bottom of the skin imperfection 200a. The field of view for the optics 214 is represented by the lines 212.

With reference to FIG. 4, a particular illustrative embodiment of a system of treating skin imperfections is generally designated 400. In a particular embodiment, the system 400 may be configured to perform the methods and system depicted in FIGS. 1-3. The system 500 includes a laser 530 having at least one processor 508 and a memory 510 that is accessible to the processor 508. The memory 510 includes media that is readable by the processor 508 and that stores data and program instructions that are executable by the processor 508, including a detection module 512 for detecting at least one skin imperfection based on electromagnetic reflections, a calculating module 514 for determining the width and volume of skin imperfections, an associative module to associate a skin value based on at least one skin imperfection, and a data file 518 that includes a database of skin values that correlate to a total volume of a skin imperfection of and a size of the preselected area of skin of a client 522 and historical skin values of representative clients 524. A display 550 is coupled to the system 500 and may include a liquid crystal display, a light emitting diode screen, or any other type of display suitable for this purpose may be used. Although depicted as separate components, the detection module 512, the calculating module 514, the associative module 516, or any combination thereof, may be integrated into a single software package or software applications that are compatible to interoperate with each other.

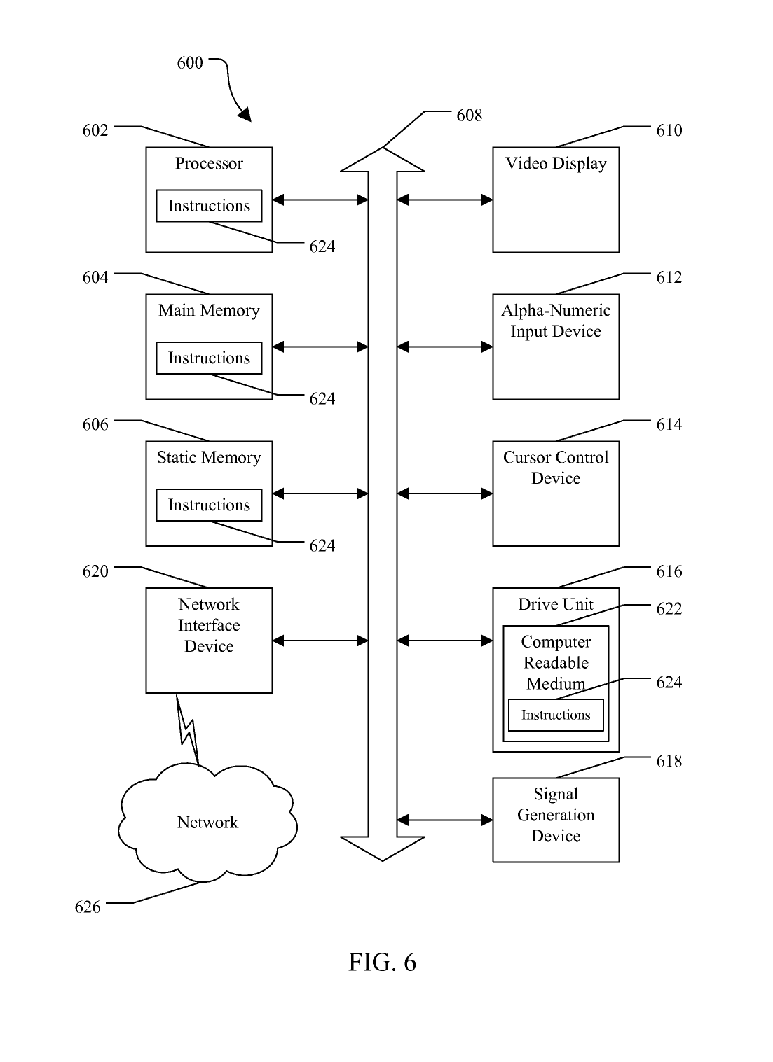

Referring to FIG. 6, an illustrative embodiment of a general computer system is shown and is designated 600. The computer system 600 may include a set of instructions that may be executed to cause the computer system 600 to perform any one or more of the methods or computer based functions disclosed herein. The computer system 600, or any portion thereof, may operate as a standalone device such as the diagnostic tool 204 or may be connected, e.g., using a network, to other computer systems or peripheral device.

In a networked deployment, the computer system may operate in the capacity of a server or a transmitter. The computer system 600 may also be implemented as or incorporated into various devices, such as the diagnostic tool 204, personal computer (PC), a tablet PC, a media device (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular embodiment, the computer system 600 may be implemented using electronic devices that provide voice, video or data communication. Further, while a single computer system 600 is illustrated, the term “system” shall also be taken to include any collection of systems or sub-systems that individually or jointly execute a set, or multiple sets, of instructions to perform one or more computer functions.

As illustrated in FIG. 6, the computer system 600 may include a processor 602, e.g., a central processing unit (CPU), a graphics-processing unit (GPU), or both.

Moreover, the computer system 600 may include a main memory 604 and a static memory 606 that can communicate with each other via a bus 608. As shown, the computer system 600 may further include a video display unit 610, such as a liquid crystal display (LCD), a light emitting diode (LED), a flat panel display, a solid-state display, or a cathode ray tube (CRT). Additionally, the computer system 600 may include an input device 612, such as a keyboard, and a cursor control device 614, such as a mouse. The computer system 600 can also include a disk drive unit 616, a signal generation device 618, such as a speaker or remote control, and a network interface device 620.

In a particular embodiment, as depicted in FIG. 6, the disk drive unit 616 may include a computer-readable medium 622 in which one or more sets of instructions 624, e.g. software, can be embedded. Further, the instructions 624 may embody one or more of the methods or logic as described herein. In a particular embodiment, the instructions 624 may reside completely, or at least partially, within the main memory 604, the static memory 606, and/or within the processor 602 during execution by the computer system 600. The main memory 604 and the processor 602 also may include computer-readable media.

In an alternative embodiment, dedicated hardware implementations, such as application specific integrated circuits, programmable logic arrays and other hardware devices, can be constructed to implement one or more of the methods described herein. Applications that may include the apparatus and systems of various embodiments can broadly include a variety of electronic and computer systems. One or more embodiments described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

In accordance with various embodiments of the present disclosure, the methods described herein may be implemented by software programs executable by a computer system. Further, in an exemplary, non-limited embodiment, implementations can include distributed processing, component/object distributed processing, and parallel processing.

The present disclosure contemplates a computer-readable medium that includes instructions or receives and executes instructions responsive to a propagated signal, so that a device connected to a network can communicate voice, video or data over the network. Further, the instructions may be transmitted or received over the network via the network interface device.

The term medium includes a single medium or multiple media, such as a centralized or distributed database, and/or associated caches and servers that store one or more sets of instructions. The term medium shall also include any medium that is capable of storing, encoding or carrying out a set of instructions for execution by a processor or that cause a computer system to perform any one or more of the methods or operations disclosed herein.

In a particular non-limiting, exemplary embodiment, the computer-readable medium can include a solid-state memory such as a memory card or other package that houses one or more non-volatile read-only memories. Further, the computer-readable medium can be a random access memory or other volatile re-writable memory. Additionally, the computer-readable medium can include a magneto-optical or optical medium, such as a disk or tapes or other storage device to capture carrier wave signals such as a signal communicated over a transmission medium. A digital file attachment to an email or other self-contained information archive or set of archives may be considered an equivalent to a tangible storage medium. Accordingly, the disclosure is considered to include any one or more of a computer-readable medium and other equivalents and successor media, in which data or instructions may be stored.

In accordance with various embodiments, the methods described herein may be implemented as one or more software programs running on a computer processor. Dedicated hardware implementations including, but not limited to, application specific integrated circuits, programmable logic arrays and other hardware devices can likewise be constructed to implement the methods described herein. Furthermore, alternative software implementations including, but not limited to, distributed processing or component/object distributed processing, parallel processing, or virtual machine processing can also be constructed to implement the methods described herein.

It should also be noted that instructions and data of the disclosed methods may optionally be stored on a tangible storage medium, such as: a magnetic medium, such as a disk or tape; a magneto-optical or optical medium, such as a disk; or a solid state medium, such as a memory card or other package that houses one or more read-only (non-volatile) memories, random access memories, or other re-writable (volatile) memories. The software may also utilize a signal including computer instructions. A digital file attachment to e-mail or other self-contained information archive or set of archives is considered a distribution medium equivalent to a tangible storage medium.

Accordingly, the disclosure is considered to include a tangible storage medium or distribution medium as listed herein, and other equivalents and successor media, in which the software implementations herein may be stored.

Although the present specification describes components and functions that may be implemented in particular embodiments with reference to particular standards and protocols, the invention is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. §1.52(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A method of treating skin imperfections, the method comprising:
scanning a preselected area of skin of a client using a diagnostic tool;
detecting a first edge and a second edge of at least one skin imperfection within the preselected area using the diagnostic tool;
detecting a surficial depth of the at least one skin imperfection using the diagnostic tool;
determining a width of the at least one skin imperfection using a spatial difference between the first edge relative to the second edge;
calculating a volume of the at least one skin imperfection using the depth and width of the at least one skin imperfection;
associating a skin value to the client based on a total volume of the at least one skin imperfection and a size of the preselected area of the skin of the client;
manually selecting a first unadulterated ingredient from a plurality of unadulterated ingredients based on the skin value;
manually selecting a second unadulterated ingredient from the plurality of ingredients based on the skin value; and
compounding the first unadulterated ingredient and the second unadulterated ingredient to form a customized product at the point of sale, wherein the compounding is manually completed by a technician.

2. The method of claim 1, wherein the diagnostic tool includes a photodetector and a laser that emits coherent light, the photodetector responsive to the electromagnetic reflections from the skin.

3. The method of claim 1, further comprising treating a cause of the at least one skin imperfection of the client by applying a portion of the customized product to an affected area of the at least one skin imperfection at the point of sale.

4. The method of claim 1, further comprising aiming the diagnostic tool at the preselected area of skin and activating a trigger.

5. The method of claim 4, wherein the diagnostic tool further comprising a memory for storing the skin value of the client.

6. The method of claim 5, further comprising displaying the skin value to the client.

7. The method of claim 5, further comprising referencing the diagnostic tool at a start of each scanning session.

8. The method of claim 1, wherein the first unadulterated ingredient is one of alpha lipoic acid, dimethylaminoethenol, vitamin A, vitamin C, and vitamin E.

9. The method of claim 1, wherein the second unadulterated ingredient is one of retinol, alpha and beta hydroxyl acids.

10. A method of treating skin imperfections, the method comprising:
manually selecting a first unadulterated ingredient;
manually selecting a second unadulterated ingredient; and
compounding the first unadulterated ingredient and the second unadulterated ingredient to form a customized product at the point of sale, wherein the compounding is manually completed by a technician.

11. A system of treating skin imperfections, the system comprising:
a diagnostic tool to scan a preselected area of skin of a client to detect at least one skin imperfection;
an associative module to associate a skin value to the client based on the at least one skin imperfection;
a first unadulterated ingredient of alpha lipoic acid, dimethylaminoethenol, vitamin A, vitamin C, and vitamin E, or any combination thereof, selected based on the skin value;
a second unadulterated ingredient of retinol, alpha and beta hydroxyl acids, or any combination thereof, selected based on the skin value; and
a customized product at the point of sale compounded using a selected first unadulterated ingredient and a selected second unadulterated ingredient, wherein the product is compounded manually by a technician.

12. The system of claim 11, wherein the diagnostic tool comprises:
a laser that emits coherent light to scan a preselected area of skin; and
a photodetector responsive to electromagnetic reflections by the laser that are reflected from a surface of the skin, wherein the electromagnetic reflections detect at least one skin imperfection.

13. The system of claim 12, wherein the associative module associates the skin value to the client based on the electromagnetic reflections from the at least one skin imperfection.

14. The system of claim 11, further comprising a third unadulterated ingredient adapted to serve as a delivery vehicle for the first and second unadulterated ingredients to penetrate down to a deep layer of a dermis of the client.

15. The system of claim 14, wherein the diagnostic tool further comprising a memory for storing the skin value of the client.

16. The system of claim 15, further comprising a display for displaying a representation of the electromagnetic reflections to the client.

17. The system of claim 11, further comprising a sponsor entity associated with a skin care product provider entity, the sponsor entity providing business-conducting space to the technician.

18. The system of claim 11, wherein the customized product further comprising at least one pure, fresh, preservative-free, anti-aging super nutrient of proven therapeutic effectiveness in topical application, and appropriate vehicles for topical delivery of the super nutrients.

19. The system of claim 16, further comprising means to compare the electromagnetic reflections to previously stored data for the client to determine results of the customized product.

20. The system of claim 12, wherein the diagnostic tool is further configured to detect a surficial depth of the at least one skin imperfection based on a known speed of the coherent light and measuring a time interval between an initial signal received representing a normal surface of the skin, and a subsequent signal received representing a depth of the skin imperfection.
